(19) United States
(12) Patent Application Publication
(10) Pub. No.: US 2024/0197603 A1
(43) Pub. Date: Jun. 20, 2024

(54) METHODS AND COMPOSITIONS FOR TREATING HYPERPIGMENTATION DISORDERS

(71) Applicants: INSERM (INSTITUT NATIONAL DE LA SANTÉ ET DE LA RECHERCHE MÉDICALE), Paris (FR); UNIVERSITÉ COTE D'AZUR, Nice (FR)

(72) Inventors: Robert BALLOTTI, NICE (FR); Corine BERTOLOTTO, Nice (FR)

(21) Appl. No.: 18/553,334

(22) PCT Filed: Mar. 30, 2021
(86) PCT No.: PCT/IB2021/000260

§ 371 Date: Sep. 29, 2023

(51) Int. Cl. A61K 8/49; A61K 31/423; A61P 17/00; A61Q 19/02

(52) U.S. Cl. CPC A61K 8/49; A61K 31/423; A61P 17/00; A61Q 19/02

(57) ABSTRACT

The pigmentation disorders alter people's quality of life, the effectiveness of treatments is limited resulting in unsatisfactory outcomes, and there is a high therapeutic demand. The inventors tested BCH and JPH203 on a more physiological model, using reconstructed human epidermis and confirmed a strong inhibition of pigmentation demonstrating the clinical potential of SLC7A5 inhibition and positioning BCH and JPH203 as depigmenting agents suitable for cosmetic or dermatologic intervention in hyperpigmentation diseases. Thus, the invention relates to a method for treating hyperpigmentation disorder in a subject in need thereof comprising administering said subject with a therapeutically effective amount of JPH203.

### FIELD OF THE INVENTION

The invention is in the field of cosmetics and/or dermatology. It finds particularly advantageous application in the field of skin care and especially for the treatment of hyperpigmentation including skin depigmentation or skin bleaching.

### BACKGROUND OF THE INVENTION

The melanin pigments, which are responsible for the color of the skin in humans, are synthesized by melanocytes in the epidermis. Therefore, the molecular mechanisms that control the growth and differentiation of melanocytes have an influence on skin pigmentation.

Melanin synthesis, or melanogenesis, is tightly regulated by tyrosinase (TYR), a melanocyte-specific enzyme, that catalyzes many reactions, including the rate-limiting step of melanogenesis, the hydroxylation of tyrosine to L-3,4-dihydroxyphenylalanine (L-DOPA). Other enzymes are involved in melanogenesis. For example, tyrosinase-related protein-1 (TYRP1) has a DHICA oxidase activity and the enzyme coded by DCT has a Dopachrome Tautomerase activity. TYRPI and DCT are involved in the synthesis of eumelanin, the black-brown melanin responsible for photo-protection against noxious effect of UV radiations (Miyamura et al., 2007).

Melanogenesis occurs in specialized organelles called melanosomes. They are related to lysosomes but contain several specific proteins that allow biogenesis and specification of these vesicles, such as PMEL17, MART1, GPR143, SLC45A2, SLC24A5 and many others (Kondo and Hearing, 2011). The melanocytes are equipped with a specific transport machinery of intracellular vesicles, involving RAB27A, MLPH and MYOVa that allows melanosomes to accumulate at the ends of melanocyte dendrites. This process favors melanosome transfer to neighboring keratinocytes and uniform skin pigmentation (Hume and Seabra, 2011; Sitaram and Marks, 2012).

To date, more than 250 genes have been involved directly or indirectly in the control of skin pigmentation, in humans (Baxter et al., 2019). Among these genes, MITF plays a key role in these developmental processes, because it is at the crossroads of all the signaling pathways involved in the development of melanocytes. MITF is a transcription factor that regulates the expression of many genes involved in the proliferation, survival and migration of melanocytes, such as CDK2, BCL2 and MET (non-exhaustive list). Other MITF target genes are directly involved in melanocyte differentiation processes. MITF controls the expression of all the genes encoding the proteins evoked above that are required for melanin synthesis, melanosome biogenesis, and melanosome transport. MITF ensures the coordinated regulation of all these processes that are necessary for optimal differentiation and physiological skin pigmentation (Cheli et al., 2010; Goding and Arnheiter, 2019). The pigmentation disorders alter people's quality of life, the effectiveness of treatments is limited resulting in unsatisfactory outcomes, and there is a high therapeutic demand.

Here, the inventors aimed at identifying previously unreported genes involved in pigmentation that might be molecular targets for depigmenting agents to be used in the treatment of hyperpigmentation diseases, such as melasma or actinic lentigo. As a large portion of MITF target genes identified so far does not have known functions, their involvement in melanocyte differentiation and pigment synthesis deserves to be evaluated.

Combined bioinformatics analysis of public ChIP-Seq and transcriptomic data allowed the inventors to identify direct MITF target genes that were, to their knowledge previously unreported. Further analysis, pointed-out to SLC7A5 as a previously unreported MITF target and pigmentation gene. SLC7A5 encodes an amino-acid transporter that genetic or pharmacologic inhibition decreases melanin pigment synthesis and allows depigmentation of reconstructed pigmented epidermis, pointing-out to SLC7A5 as a potential target in the treatment of hyper-pigmentation pathologies. Thus there is a need to identify new treatments against pigmentation disorders.

### SUMMARY OF THE INVENTION

The invention relates to methods and compositions for treating hyperpigmentation disorders. In particular, the present invention is defined by claims.

### DETAILED DESCRIPTION OF THE INVENTION

Integration of ChIPseq and microarray data allowed to identify to the inventor's knowledge previously unreported MITF target genes, among which, the amino acid transporter, SLC7A5. The inventors showed that siRNA-mediated SLC7A5 knock-down decreased pigmentation in B16F10 cells, without affecting morphology nor dendricity. Treatment with the SLC7A5 inhibitors BCH, or JPH203, also decreased melanin synthesis in B16F10 cells. The inventor's findings indicated that BCH was as potent as reference depigmenting agent, Kojic Acid, but acted through a different pathway not affecting tyrosinase activity. BCH also decreased pigmentation in human MNT1 melanoma cells or normal human melanocytes. Finally, the inventors tested BCH and JPH203 on a more physiological model, using reconstructed human epidermis and confirmed a strong inhibition of pigmentation demonstrating the clinical potential of SLC7A5 inhibition and positioning BCH and JPH203 as depigmenting agents suitable for cosmetic or dermatologic intervention in hyperpigmentation diseases.

In a first aspect, the invention relates to a method for treating hyperpigmentation disorder in a subject in need thereof comprising administering said subject with a therapeutically effective amount of JPH203.

As used herein, the term “subject” refers to any mammals, such as a rodent, a feline, a canine, and a primate. Particularly, in the present invention, the subject is a human. More particularly, the subject is a human suffering from one of the hyperpigmentation disorders as describes above.

As used herein, the terms “hyperpigmentary skin disorder” or “hyperpigmentation disorder” are used interchangeably and refer to the darkening of an area of skin or nails caused by increased melanin. Hyperpigmentation is the result of either of two occurrences: (1) an abnormally high concentration of melanocytes produce melanin or (2) when melanocytes are hyperactive. Hyperpigmentation disorders can affect any part of the body including the face, hands, and neck. Hyperpigmentation disorder is selected form the group consisting of but not limited to depigmentation of the skin, skin bleaching, solar lentigines, melasma, freckles, age spots, post-acne pigmentation and post-inflammatory hyperpigmentation.

As used herein, the term “depigmentation of the skin” refers to the lightening of the skin or loss of pigment. Depigmentation of the skin can be caused by a number of local and systemic conditions. The pigment loss can be partial (injury to the skin) or complete (caused by vitiligo). It can be temporary (from tinea versicolor) or permanent (from albinism).

As used herein, the term “skin bleaching”, also known as skin lightening or skin whitening, is the practice of using chemical substances in an attempt to lighten the skin or provide an even skin color by reducing the melanin concentration in the skin. Several chemicals have been shown to be effective in skin whitening, while some have proven to be toxic or have questionable safety profiles. This includes mercury compounds which may cause neurological problems and kidney problems.

As used herein, the term “lentigo/lentigines” or “solar lentigines,” also known as a sun-induced freckle or senile lentigo, is a dark (hyperpigmented) lesion caused by natural or artificial ultraviolet (UV) light. The term “melasma” also called as pregnancy-induced melasma. It is also known as pregnancy mask or chloasma. With melasma, the pigmentation is generally symmetrical and has clearly defined edges.

As used herein, the term “freckles” refers to flat circular spots which are usually tan or light brown in colour. While freckles are an extremely common type of hyperpigmentation, they are more often seen among people with a lighter skin tone.

As used herein, the term “age spots” refers to tan, brown or black in colour. Age spots are oval in shape and the size varies from freckle size to more than 13 mm. It is also known as liver spots and they tend to develop on the face and other photo-exposed areas after the age of 40.

As used herein, the term “post acne pigmentation” refers to marks caused by acne. They can be observed in more than 60% of acne in some ethnies. In most cases pigmentary marks which are dark in colour result from an overproduction of melanin in reaction to skin inflammation at the affected area. Without proper treatment, post-acne pigmentation may take months or even years to fade off. The term “post inflammatory hyperpigmentation” refers to the marks caused by an injury or inflammation to the skin, there is an increased production of colour pigment in such conditions.

In one embodiment, the hyperpigmentation pathology is a depigmentation of the skin

In one embodiment, the hyperpigmentation pathology is a skin bleaching.

As used herein, the term “LAT-1/SLC7A5 transporter” refers a protein having the function of the cellular transport of large neutral amino acids, or called also L-type amino acids, such as phenylalanine, tyrosine, leucine, arginine, tryptophan. The LAT-1 transporter is a transmembrane transporter. The LAT-1 transporter is encoded by the SLC7A5 gene. The LAT-1 transporter is expressed in a large number of cell types ranging T cells, cancer cells or even endothelial cells of the brain.

As used herein, the term “inhibitor of LAT-1/SLC7A5 transporter” has its general meaning in the art, and refers to a compound which has the capability of reducing or suppressing selectively the activity or expression of the LAT-1/SLC7A5 transporter. In the context of the invention, the compound inhibits the interaction of LAT-1/SLC7A5 transporter.

As used herein the term, “JPH203” refers to a chemical molecule knows as O-[(5-amino-2-phenyl-7-benzoxazolyl)methyl]-3,5-dichloro-L-tyrosine. The case number is 1037592-40-7 and the molecule is represented by the following formula:

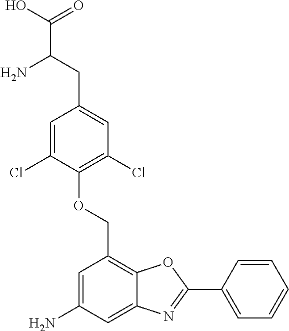 [SMILES: Nc1cc(COc2c(Cl)cc(CC(N)C(=O)O)cc2Cl)c2oc(-c3ccccc3)nc2c1]

As used herein, the term “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “topical application” refers to the fact of applying or spreading the compositions according to invention on the surface of the skin,

As used herein the term “cosmetically or pharmaceutically acceptable”, that the composition according to the invention is suitable for coming into contact with the body and more particularly the skin without causing toxicity reactions or intolerance.

As used herein the terms “administering” or “administration” refer to the act of injecting or otherwise physically delivering a substance as it exists outside the body (e.g., an inhibitor of LAT-1/SLC7A5 transporter, i.e. JPH203) into the subject, such as by topical, intradermal, mucosal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of an inhibitor of LAT-1/SLC7A5 transporter, i.e.. JPH203 for use in a method for the treatment of hyperpigmentation pathology at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitor of LAT-1/SLC7A5 transporter, i.e.. JPH203 as described above may be combined with pharmaceutically acceptable excipients, and optionally sustained-release matrices, such as biodegradable polymers, to form pharmaceutical compositions.

In a second aspect, the invention relates to a composition for use in the treatment of hyperpigmentation disorder comprising an effective amount of JPH203 or a cosmetically or pharmaceutically salt acceptable of that one.

In particular, the composition comprises from 0.5% to 20% by weight of inhibitor of the LAT-1/SLC7A5 transporter relative to the total weight of the composition.

In particular, the composition comprises from 0.5% to 20% by weight of. JPH203 relative to the total weight of the composition.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to one or more of the following agents: solvents such as olive oil, olive oil refined, cottonseed oil, sesame oil, sunflower seed oil, peanut oil, wheat germ oil, soybean oil, jojoba oil, evening primrose oil, coconut oil, palm oil, sweet almond oil, aloe oil, apricot kernel oil, avocado oil, borage oil, hemp seed oil, macadamia nut oil, rose hip oil, pecan oil, hazelnut oil, sasanqua oil, rice bran oil, shea butter, corn oil, camellia oil, grape seed oil, canola oil, castor oil, and combinations thereof, preferably olive oil refined, emulsifiers, suspending agents, decomposers, binding agents, excipients, stabilizing agents, chelating agents, diluents, gelling agents, thickening agent such as beeswax and/or petroleum jelly, preservatives, lubricants, absorption delaying agents, liposomes, antioxidants such as butylhydroxytoluene or butylhydroxyanisole, and the like. It also refers to a non-toxic solid, semi-solid or liquid filler, diluent, encapsulating material or formulation auxiliary of any type. The pharmaceutical compositions of the present invention for topical, subcutaneous, oral, sublingual, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise topical such as cream, oral-route forms such as tablets, gel capsules, powders, granules and oral suspensions or solutions, sublingual and buccal administration forms, aerosols, implants, subcutaneous, transdermal, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for topical, intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. The pharmaceutical composition is formulated into a topical formulation that can be directly applied to the skin, for example, a skin suffering from hyperpigmentation disorders. In one embodiment of this application, the pharmaceutical composition is formulated into an external preparation by admixing the extract according to this application with a base such as those that are well known and commonly used in the art. The person responsible for administration will, in any event, determine the appropriate dose for the individual subject. Some variation in dosage will necessarily occur depending on the condition of the subject being treated.

The composition according to the invention is present in all the dosage forms normally used in cosmetics and dermatology suitable for topical application for example: ointment, The topical formulation suitable for the pharmaceutical composition may be a gel, a patch, an embrocation, an aerosol, a spray, a paste, a foam, a drop, a serum lotion, an emulsion of more or less consistency fluid, white or colored, obtained by dispersing a fatty phase in a phase aqueous, oil-in-water emulsion (O/W) or conversely water-in-oil emulsion (W/O) milk, cream, gel, gel-emulsion, mask or anhydrous oil balm, powder. They can also be in the form of a stick. Among these preparations, creams, fluid or thick gels, or the spray are preferred whose application is simple and easy on all parts of the body. The compositions according to the invention are applied topically in an amount sufficient in humans, that is to say in an amount corresponding to the application doses usual for the type of composition considered (gel, cream, lotion, etc.). For example, in the case of a face cream, 0.5 to 3 g is applied, and in particular 1 to 2 g of cream per day in one or more exposures. For example, in the case of a body cream, 5 to 12 g, and in particular 7 to 10 g of cream are applied per day in one or more exhibitions.

The invention will be further illustrated by the following figures and examples. However, these examples and figures should not be interpreted in any way as limiting the scope of the present invention.

### FIGURES

FIG. 1. SLC7A5 silencing affects melanogenesis. Melanin content in B16F10 cells after SLC7A5 silencing. Values were normalized per amount of proteins and calculated as percentage of the control condition (siCt). Values are mean±SD from three different experiments. **** p<0.0001.

FIG. 2. Effects of JPH203 on melanin synthesis. Quantification of melanin content in B16F10 cells treated with various doses of JPH203. Values were normalized per amount of proteins and calculated as percentage of the basal condition. Values are mean±SD from three different experiments. * p<0.05, ** p<0.01.

### EXAMPLE

### Material & Methods

### Cell Culture and Chemicals

Mouse B16F10 and Human 501Mel melanoma cells were cultured in Dulbecco's modified Eagle's medium (DMEM) supplemented with 7% Fetal Bovine Serum and 1% Penicillin/Streptomycin. Human MNT1 melanoma cells were cultured as described (Yasumoto et al., 2004). Normal Human Melanocytes were isolated from foreskin obtained after phimosis surgery. Patient consent was not required because French laws consider tissue left over from postectomy as discarded material. Melanocytes were cultured in MCDB medium as described (Bonet et al., 2017). Cells were tested every 4 weeks for mycobacterial presence. BCH (Tocris, France) and Kojic acid (Sigma, France) were resuspended in water at 50 mM, and JPH203 (Selleckchem, France) was resuspended in DMSO at a 50 mM stock solution.

### Cell Viability

Cell viability was evaluated using CellTiter Aqueous One Solution Cell Proliferation Assay (Promega, France) as recommended by the supplier.

### Gene Silencing and Induction of Pigmentation

Cells were transfected with 50 nM siRNAs to SLC7A5, MITF or control siRNAs (ON-TARGETplus, Dharmacon) using HiPerfect transfection reagent (Qiagen, France). When indicated, Forskolin (20 μM) and IBMX (100 μM) were added to induce pigmentation on B16F10 cells. 48 hours later, medium was removed, and cells were used for western blot, quantitative PCR, melanin content analyses, immunofluorescence or brightfield imaging.

### Protein Expression

Proteins were analyzed by SDS-PAGE and transferred on PVDF membranes (Sigma, France). The antibodies used were as follow: beta-actin (ab8226), MITF (ab12039) and Tyrosinase (ab738) from Abcam, Rab27a (#69295) and HSP90 (#4874) from Cell Signaling Technologies, SLC7A5 (sc-374232) and TYRPI (sc-166857) from Santa Cruz. Signals were detected with horseradish peroxidase conjugated secondary antibodies using ECL detection kit and quantified by digital imaging (Fuji LAS4000).

### Melanin Content

Cells were detached with 0.05% Trypsin-EDTA solution (Thermo Scientific, France) and solubilized in NaOH 0.5N. Optical density was measured at 405 nm using melanin standard as a reference. Melanin content was normalized to the protein content.

### Immunofluorescence and Confocal Analysis

Cells were fixed with Paraformaldehyde 4%, permeabilized with Triton 0.1% and incubated with anti-TYRPI antibody (Santa Cruz, sc-58438). Fluorescent signal was revealed using Alexa Fluor 594 labeled secondary antibody (Thermo Scientific, France). Images were obtained using confocal Nikon A1R microscope (40× oil immersion lens).

Real-Time Quantitative PCR Real-time quantitative PCR was carried out with SYBR Green using a StepOne Real-Time PCR System (Thermo Scientific, France). Results were normalized using GAPDH. Primer sequences are available upon request.

### L-DOPA Activity

Proteins were resuspended in 0.1M Sodium Phosphate pH 6.8, supplemented with 5 mM L-DOPA. After incubation for 1 hour at 37° C., optical density was read at 475 nm.

### Reconstructed Human Pigmented Epidermis

Phototype VI Reconstructed Human Pigmented Epidermis (Sterlab, France) were treated with BCH or vehicle for 7 days. 10 μm cryosections were used to visualize melanin using Fontana-Masson stain kit (Interchim, France). Images were taken with a Nikon microscope and a 20× lens. Melanin content was quantified after solubilization of epidermis in Solvable® solution (PerkinElmer, France), and melanin content was quantified as described above.

### Statistical Analyses

All data are presented as mean±SD. A one-way ANOVA test was used for all the experiments, followed by a Dunnett's multiple comparisons test.

### Data Availability

Datasets related to this article can be found at [https://www.ncbi.nlm.nih.gov/geo/query/acc.cgi?acc-GSE61967], hosted at GEO Omnibus (Laurette et al., 2015) and at [https://portals.broadinstitute.org/ccle/data], hosted by the BROAD institute.

### Results

### Identification of Direct MITF Target Genes

First, to identify genes bound by MITF and expression of which covaries with MITF, we combined analysis of the MITF ChIP seq experiment (Laurette et al., 2015) with that of the CCLE melanoma panel (Broad Institute). This analysis identified 1068 pics in 946 genes. Gene ontology analysis of these genes showed an enrichment of terms associated with melanin synthesis (Data not shown), strengthening the role of MITF in positive regulation of pigmentation genes.

Among the 946 genes, we searched for those that are down-regulated by MITF siRNA in both 501Mel human melanoma cells and B16F10 mouse melanoma cells. We found 5 genes (Data not shown) that fulfil all these criterions. Among these genes, TYR, RAB27A and MLPH were already described as involved in pigmentation. Two additional genes were identified, ST3GAL6 and SLC7A5 whose functions in pigmentation were not documented so far.

First round of functional analysis using siRNA to St3gal6 and Slc7a5 showed that St3gal6 silencing inhibited proliferation of B16F10 melanoma cells (Data not shown), while the inhibition of Slc7a5 expression had no significant effect on cell proliferation (Data not shown). Therefore, Slc7a5 was selected for further studies.

### Validation of SLC7A5 as a MITF Target

A UCSC browser image capture of the SLC7A5 promoter region from MITF (Black) (Laurette et al., 2015), H3K27ac and H3K4me3 (purple) (Ohanna et al., 2018) ChIP-seq experiments (Data not shown) confirmed that MITF bound to the SLC7A5 promoter, and overlap histone activating epigenetic marks. Then, transfection of B16F10 melanoma cells with MITF siRNA showed a decrease in Mitf and Slc7a5 expression, both at messenger (Data not shown) and protein level (Data not shown) demonstrating that Slc7a5 is a Mitf target gene. MITF silencing also decreased SLC7A5 expression in 501Mel cells (Data not shown).

### SiRNA-Mediated Slc7a5 Knock-Down Inhibits Melanogenesis in B16F10 Cells

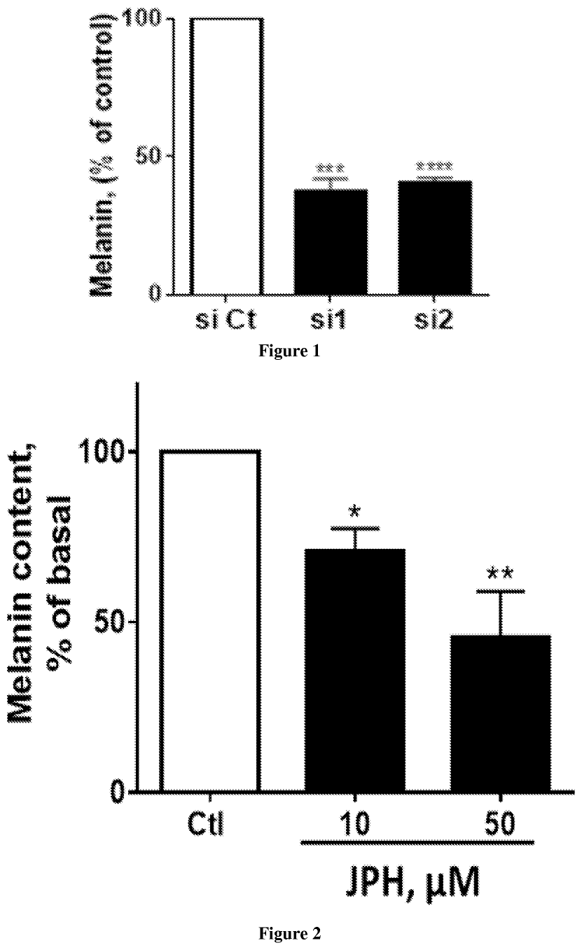

Then we studied the effects of Slc7a5 silencing on pigmentation in B16F10 melanoma cells. Cells transfected with control (siCt) or Slc7a5 (siSlc7a5) siRNA were incubated with CAMP-elevating agents (Forskolin, 20 μM, IBMX 100 μM) to increase melanogenesis. 48 hours later, western blot analysis confirmed the strong inhibition of Slc7a5 expression at protein level, by 2 different siSlc7a5 (Data not shown). On bright field images (Data not shown), a clear inhibition of cells pigmentation was seen after Slc7a5 silencing. This observation was confirmed by measurement of melanin content (FIG. 1). Both siRNA directed against Slc7a5 resulted in a 60% decrease in melanin content. Unexpectedly, Slc7a5 silencing led to a slight and barely significant increase in Tyrosinase (Tyr) and Det, while the effect was much more pronounced for Tyrp1 expression. No significant effect was observed for Rab27a expression (Data not shown). qPCR analyses demonstrated that siSlc7A5 effectively inhibited Slc7a5 mRNA level, but did affect significantly the expression of Tyr, Tyrp1, Dct and Rab27a (Data not shown). Immunofluorescence studies of Tyrp1 expression showed that Slc7a5 silencing did not affect drastically cells morphology or dendricity and confirmed the increased expression of Tyrp1 (Data not shown). As Tyrp1 expression is clearly localised at the cell periphery, Slc7a5 silencing does not seem to affect melanosome transport.

### Pharmacological Inhibition of SLC7A5 Decreases Melanin Content in B16F10 Cells

Next, we investigated the effects of pharmacological inhibitors of SLC7A5, BCH, and JPH203 that is now in clinical trial as an anti-cancer drug.

First, dose response experiments showed that BCH, up to 10 mM did not affect significantly B16F10 cell viability (Data not shown). Evaluation of melanin content showed a significant inhibition of melanin production in B16F10 cells, by BCH at 5 mM (25%), and 10 mM (60%) (Data not shown). Bright field images confirmed that BCH inhibited pigmentation at 10 mM (Data not shown). Immunofluorescence with Tyrp1 antibody showed that BCH at 10 mM did not obviously alter morphology of the cells (Data not shown). As it was the case with the siRNA directed against Slc7a5, we observed a consistent increase in Tyrp1 labelling. Western blot analysis confirmed a dose-dependent increase in Tyrp1 expression in response to BCH, while other melanogenesis proteins, such as Tyrosinase or Rab27a, were not affected consistently by BCH. BCH did not affect Slc7a5 expression (Data not shown).

Strengthening this observation, JPH203 at 10 and 50 μM, also efficiently inhibited pigmentation in B16F10 cells at doses that did not inhibit cell proliferation (FIG. 2). Together these observations confirmed the involvement of Slc7a5 in melanogenesis and the possible use of pharmacological inhibition of Slc7a5 as depigmenting strategy.

### Comparison of BCH and Kojic Acid Effects on Pigmentation

Next, we compared the effects of BCH to that of a well-known reference depigmenting agent, Kojic Acid. Dose response experiments confirmed the inhibition of melanogenesis by Kojic acid (Data not shown), at doses that did not affect cell proliferation (Data not shown). At 1 mM, we observed an inhibition of 60% of melanin content. This effect is comparable to that observed in B16F10 cells in response to BCH. Furthermore, treatment of B16F10 cells with Kojic Acid (1 mM), inhibited tyrosinase activity, while BCH (10 mM) did not (Data not shown). Then, when added directly in cell lysates during the DOPA oxidase assay (Data not shown), Kojic Acid, but not BCH, inhibited tyrosinase activity. Therefore, we can conclude that BCH is as potent as Kojic Acid to inhibit melanogenesis in B16F10 melanoma cells, and that BCH does not inhibit tyrosinase activity.

### BCH Inhibited Pigmentation in MNT1 Human Melanoma Cells, Normal Human Melanocytes, and Reconstructed Human Pigmented Epidermis

Next, we verified the effects of BCH on human cells and on more physiological models such as normal melanocytes and reconstructed epidermis. First, using highly pigmented human melanoma cells, MNT1 (Data not shown) and pigmented normal human melanocytes (NHM) (Data not shown), we confirmed that in both cell types, BCH significantly decreased melanin production. Then using reconstructed pigmented epidermis containing both melanocytes and keratinocytes, we clearly observed that BCH treatment inhibited pigmentation, as shown by macroscopic images (Data not shown) and confirmed by bright field microscopic images (Data not shown) of the epidermis. Additionally, Fontana Masson staining of epidermis sections demonstrated the absence of pigmented melanocytes in reconstructed epidermis exposed to BCH (Data not shown). Finally, the quantification of melanin content confirmed a statistically significant decrease in melanin content in reconstructed epidermis treated with BCH (Data not shown).

### Discussion

Pathologies associated with melanocytes dysfunction can lead to depigmentation as in vitiligo or hyperpigmentation such as age spots (actinic lentigo) or melasma. These pigmentary defects do not endanger the lives of those affected but have a significant psychological impact. Until now, only heavy dermatological approaches have shown a real effectiveness in the treatment of pigmentary pathologies, vitiligo, melasma and actinic lentigo. The cosmetic approaches already used in the context of hyperpigmentation pathologies mainly target tyrosinase, a key enzyme in melanin synthesis, and/or the transfer of melanin to keratinocytes. These treatments show low efficiency.

In this study, using a very stringent bioinformatic analyses, we identified previously unreported MITF target genes ST3GAL6 and SLC7A5, function of which in pigmentation has never been studied. Of course, the list generated by our analyses does not contain all the known MITF target genes but can be easily increased by applying more tolerant filters.

SLC7A5 encodes for a member (LAT1) of the L-Type amino-acid transporter family that is specialized in the transport of histidine, tryptophan and tyrosine, in addition to neutral amino-acids (Singh and Ecker, 2018). Slc7a5 silencing barely affected proliferation of B16F10 melanoma cells but promoted a strong and reproducible inhibition of melanogenesis.

To understand the key role of SLC7A5 in the transport of amino-acids, thyroid hormones and metabolites, as well as its involvement in cancer cell survival/proliferation, huge efforts have been made to identify SLC7A5 inhibitors (Wang and Holst, 2015). To date, dozens of such inhibitors have been validated.

Testing BCH, the first in class SLC7A5 inhibitor (Kim et al., 2008) and JPH203, an inhibitor in clinical trial (Oda et al., 2010), we have been able to demonstrate that both inhibitors efficiently inhibited pigmentation in B16F10 melanoma cells.

Concerning the mechanism by which the inhibition of Slc7a5 affected melanogenesis, we did not observe an inhibition of the expression of melanogenic enzymes, Tyr, Tyrp1 and Dct. On the contrary, we observed that both genetic and pharmacological inhibition of Slc7a5 induces an increase in Tyrp1 expression. As there no change in mRNA level was observed, we can suggest that Slc7a5 inhibition impaired the targeting of Tyrp1 to proteasome or lysosome degradation compartments, as reported for tyrosinase (Watabe et al., 2004, Fujita et al., 2009). Our data also ruled out the possibility that BCH inhibited directly the tyrosinase activity.

SLC7A5 transports tyrosine, the key substrate for melanin synthesis. Therefore, SLC7A5 inhibition might reduce cellular tyrosine level, even though tyrosine is not an essential amino acid, melanocytes cannot synthetize tyrosine from phenylalanine. Furthermore, as melanogenesis takes place in melanosomes, tyrosine must be transported into these organelles. Tyrosine transport activity has been identified, but the nature of the transporter remains elusive. Proteomic analysis of melanosomes (Basrur et al., 2003) and immunoprecipitation of intact melanosomes (Data not shown) both failed to validate the presence of SLC7A5 in the organelles.

Additionally, SLC7A5 was reported to regulate the efflux of L-glutamine out of cells and the transport of L-leucine into the cell. Through this activity SLC7A5 plays a key role in the control of mTOR and autophagy (Nicklin et al., 2009). Interestingly, autophagy was described to regulate melanogenesis, as its activation leads to melanosome degradation and inhibition of melanin synthesis (Ho and Ganesan, 2011). Therefore, it can be proposed that SLC7A5 inhibition activates autophagy that is responsible for the inhibition of pigmentation. We assessed this hypothesis by combining autophagy inhibitors (3-MA or Chloroquine) with BCH, or SLC7A5 siRNA. However, autophagy inhibitors strongly potentiate the anti-proliferative effects of SLC7A5 inhibition (Data not shown), preventing reliable interpretation of the results. Nevertheless, BCH at 5 and 10 mM induced a conversion of LC3-I to LC3-II demonstrating an activation of autophagy and validating the inhibition of Slc7a5 by BCH (Data not shown).

Finally, we have been able to show that BCH prevented pigmentation in two different human cell models. BCH also inhibited pigmentation in reconstructed human epidermis, without affecting epidermis structure or keratinisation, predicting no major adverse effects.

Taken together, these data indicate that SLC7A5 plays an important role in melanin production and is amenable to pharmacological targeting for treatment of hyperpigmentation conditions.

### REFERENCES

Throughout this application, various references describe the state of the art to which this invention pertains. The disclosures of these references are hereby incorporated by reference into the present disclosure.
Basrur V, Yang F, Kushimoto T, Higashimoto Y, Yasumoto K, Valencia J, et al. (2003) Proteomic analysis of early melanosomes: identification of novel melanosomal proteins. J Proteome Res 2:69-79.
Baxter L L, Watkins-Chow D E, Pavan W J, Loftus S K (2019) A curated gene list for expanding the horizons of pigmentation biology. Pigment Cell Melanoma Res 32:348-58.
Bonet C, Luciani F, Ottavi J F, Leclerc J, Jouenne F M, Boncompagni M, et al. (2017) Deciphering the Role of Oncogenic MITFE318K in Senescence Delay and Melanoma Progression. J Natl Cancer Inst 109.
Cheli Y, Ohanna M, Ballotti R, Bertolotto C (2010) Fifteen-year quest for microphthalmia-associated transcription factor target genes. Pigment Cell Melanoma Res 23:27-40.
Fujita H, Motokawa T, Katagiri T, Yokota S, Yamamoto A, Himeno M, et al. (2009) Inulavosin, a melanogenesis inhibitor, leads to mistargeting of tyrosinase to lysosomes and accelerates its degradation. J Invest Dermatol 129:1489-99.
Goding C R, Arnheiter H (2019) MITF-the first 25 years. Genes Dev 33:983-1007.
Ho H, Ganesan A K (2011) The pleiotropic roles of autophagy regulators in melanogenesis. Pigment Cell Melanoma Res 24:595-604.
Hume A N, Seabra M C (2011) Melanosomes on the move: a model to understand organelle dynamics. Biochem Soc Trans 39:1191-6.
Kim C S, Cho S H, Chun H S, Lee S Y, Endou H, Kanai Y, et al. (2008) BCH, an inhibitor of system L amino acid transporters, induces apoptosis in cancer cells. Biol Pharm Bull 31:1096-100.
Kondo T, Hearing V J (2011) Update on the regulation of mammalian melanocyte function and skin pigmentation. Expert Rev Dermatol 6:97-108.
Laurette P, Strub T, Koludrovic D, Keime C, Le Gras S, Seberg H, et al. (2015) Transcription factor MITF and remodeller BRG1 define chromatin organisation at regulatory elements in melanoma cells. Elife 4.
Miyamura Y, Coelho S G, Wolber R, Miller S A, Wakamatsu K, Zmudzka B Z, et al. (2007) Regulation of human skin pigmentation and responses to ultraviolet radiation. Pigment Cell Res 20:2-13.
Nicklin P, Bergman P, Zhang B, Triantafellow E, Wang H, Nyfeler B, et al. (2009) Bidirectional transport of amino acids regulates mTOR and autophagy. Cell 136:521-34.
Oda K, Hosoda N, Endo H, Saito K, Tsujihara K, Yamamura M, et al. (2010) L-type amino acid transporter 1 inhibitors inhibit tumor cell growth. Cancer Sci 101:173-9.
Ohanna M, Cerezo M, Nottet N, Bille K, Didier R, Beranger G, et al. (2018) Pivotal role of NAMPT in the switch of melanoma cells toward an invasive and drug-resistant phenotype. Genes Dev 32:448-61.
Singh N, Ecker G F (2018) Insights into the Structure, Function, and Ligand Discovery of the Large Neutral Amino Acid Transporter 1, LAT1. Int J Mol Sci 19.
Sitaram A, Marks M S (2012) Mechanisms of protein delivery to melanosomes in pigment cells. Physiology (Bethesda) 27:85-99.
Wang Q, Holst J (2015) L-type amino acid transport and cancer: targeting the mTORC1 pathway to inhibit neoplasia. Am J Cancer Res 5:1281-94.
Watabe H, Valencia JC, Yasumoto K, Kushimoto T, Ando H, Muller J, et al. (2004) Regulation of tyrosinase processing and trafficking by organellar pH and by proteasome activity. J Biol Chem 279:7971-81.
Yasumoto K, Watabe H, Valencia JC, Kushimoto T, Kobayashi T, Appella E, et al. (2004) Epitope mapping of the melanosomal matrix protein gp100 (PMEL17): rapid processing in the endoplasmic reticulum and glycosylation in the early Golgi compartment. J Biol Chem 279:28330-8.

1. A method for treating hyperpigmentation disorder in a subject in need thereof comprising administering to said subject a therapeutically effective amount of JPH203.

2. The method according to claim 1 wherein the hyperpigmentation disorder is a depigmentation of skin.

3. The method according to claim 1 wherein the hyperpigmentation disorder is skin bleaching.

4. A composition formulated for use in the treatment of a hyperpigmentation disorder comprising a therapeutically effective amount of JPH203 or a cosmetically or pharmaceutically acceptable salt thereof.

5. The composition according to claim 4, wherein the composition is formulated for topical application.

6. The composition according to claim 4, wherein the hyperpigmentation disorder is or includes skin bleaching and/or depigmentation of skin.
